(19) United States
(12) Patent Application Publication
(10) Pub. No.: US 2012/0309100 A1
(43) Pub. Date: Dec. 6, 2012

(54) Sample preparation for isotopic analysis without an oxidizer

(76) Inventors: Gregor Hsiao, San Jose, CA (US); Joshua T. Oen, Fremont, CA (US)

(21) Appl. No.: 13/134,135

(22) Filed: May 31, 2011

(51) Int. Cl. G01N 25/00; G01N 30/02; G01N 21/59; G01N 33/00

(52) U.S. Cl. 436/155; 422/83; 73/25.01; 73/23.35; 356/437

(57) ABSTRACT

A sample preparation chamber for gas analysis that includes a heating element within is provided. The heating element is maintained at an elevated temperature, such that organic compounds that may be present in an input gas sample are removed via oxidation and/or thermal decomposition to provide a treated gas sample that is substantially free of organic contaminants. The treated gas sample may then be analyzed in a gas analysis instrument (e.g., an optical spectroscopic instrument, a mass spectrometer, etc.) to provide results that are free from interference due to organic contaminants. Preferably, the heating element is configured as a Ni—Cr wire. An important feature of this approach is that the heating element (and the rest of the sample preparation chamber) are not altered in operation to remove the organic compounds.

### FIELD OF THE INVENTION

This invention relates to sample preparation for gas analysis.

### BACKGROUND

The use of laser based spectroscopy has become common to measure the ratio between two isotopes of the same molecule. This requires a very high level of precision and the presence of other molecules which absorb in the same spectra region can reduce that precision. When laser based spectroscopy is applied in practice, the gaseous sample or ambient environment which is measured can often contain such other molecules. For example, measurements of carbon dioxide in the air above a wetland can be complicated by the presence of methane emitted from the same wet lands. Any process which removes water from a leaf can also remove soluble molecules such as alcohols due to azeotrope formation and similar vapor pressures. These other molecules can affect the analysis. For example, in an optical measurement, any alcohols present will have spectroscopic overlap and thus provide undesirable interference when measuring water isotopologues.

Recent research has found this problem in the case of waters extracted from plants using the cryogenic distillation technique. Often the molecules that cause interference are chemically very similar to the analyte, thus complicating the removal process. Ideally the other molecules could be removed in an in-line process to minimize sample handling. Extensive sample handling is highly likely to cause alteration of the isotopic composition of the sample. In the case of the isotopologues of water, the use of sorbent media and/or chromatography poses a particular challenge. If the media or column captures even a minute amount of the analyte it is likely to be of a different isotopic composition than the bulk analyte - - - this will in turn skew the isotopic ratio measurement. When subsequent samples are introduced, the residual captured previous analyte in the measurement system can undergo isotopic exchange with the new analyte sample and affect the next set of measurements. Some previous approaches have used activated charcoal in an attempt to remove alcohols from the water. There is no established technique for complete removal of ethanol from aqueous solutions without removing some of the water due to the formation of an ethanol/water azeotrope.

Although water samples can be especially difficult to handle, as described above, preparation methods for other kinds of samples have been considered in the art. One example is provided in U.S. Pat. No. 5,432,344, where sample preparation for isotopic analysis mass spectrometry entails the use of an oxidizer combined with elevated temperature to oxidize organic compounds to CO₂ and H₂O. In this work the oxidizer can be nickel oxide (optionally also including copper oxide). The role of the oxidizer is to provide the oxygen that is necessary for the oxidation to proceed. Thus, oxygen is depleted from the oxidizer during normal operation for sample preparation. At intervals, the oxidizer is reoxidized in the presence of an oxygen flow, in order to restore oxygen to the oxidizer. This requirement for re-oxidation of the oxidizer disadvantageously increases the cost and complexity of oxidizer-based approaches for sample preparation.

Accordingly, it would be an advance in the art to provide a simpler method of sample preparation for measurements such as isotopic ratio measurements.

### SUMMARY

In this work, a sample preparation chamber that includes a heating element within is employed. The heating element is maintained at an elevated temperature, such that organic compounds that may be present in an input gas sample are removed via oxidation and/or thermal decomposition to provide a treated gas sample that is substantially free of organic contaminants. The treated gas sample may then be analyzed in a gas analysis instrument (e.g., an optical spectroscopic instrument, a mass spectrometer, etc.) to provide results that are free from interference due to organic contaminants. Preferably, the heating element is configured as a Ni—Cr wire.

An important feature of this approach is that the heating element (and the rest of the sample preparation chamber) are not altered in operation to remove the organic compounds. Since no part of the apparatus is chemically altered in the chemical reactions that remove the organic contaminants, there is no need for any step analogous to reoxidization of an oxidizer (as described above in connection with U.S. Pat. No. 5,432,344). Operation and maintenance of the apparatus is thereby substantially simplified, which can provide significant advantages in practice.

### BRIEF DESCRIPTION OF THE DRAWINGS

FIG. 1 shows a block diagram of an embodiment of the invention.

FIGS. 2a-b show two views of an first sample preparation chamber configuration.

FIGS. 3a-b show two views of an second sample preparation chamber configuration.

FIGS. 4a-b show two views of an assembly including the second sample preparation chamber configuration.

### DETAILED DESCRIPTION

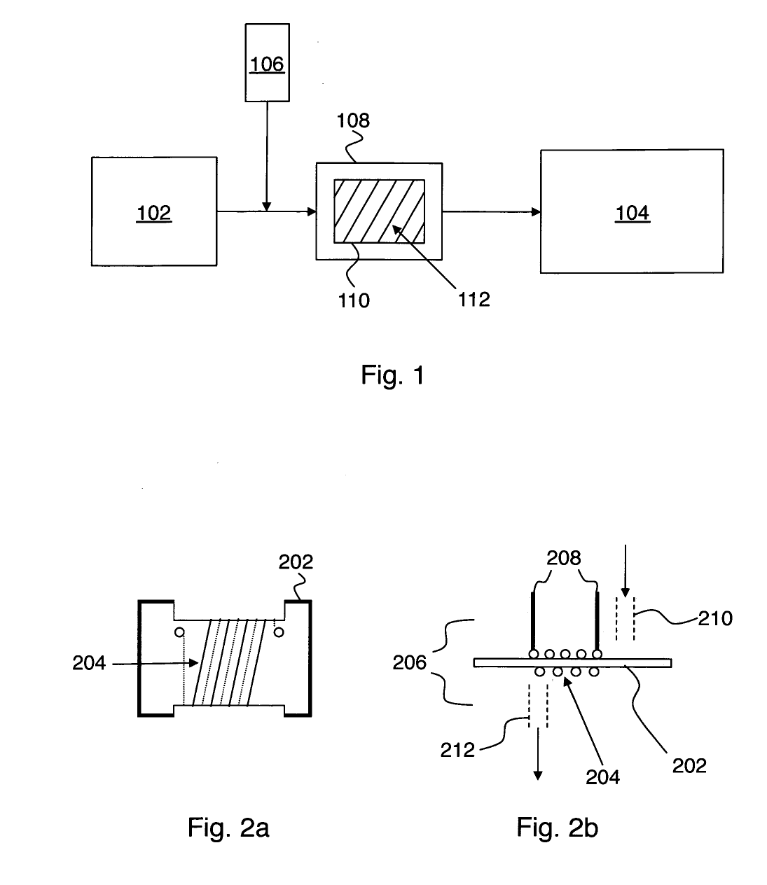

FIG. 1 shows a block diagram of an embodiment of the invention. In this example, a sample preparation chamber 108 is disposed to receive an input gas sample from a sample source 102 and to provide a treated gas sample to analysis instrument 104. In the example of FIG. 1, a heating element 112 is disposed on a support 110, which in turn is disposed within the sample preparation chamber 108. Preferably, heating element 112 is configured as a wire, however any other shape of the heating element is also possible. Practice of the invention does not depend critically on the nature of sample source 102. Any kind of gas sample can be analyzed, and sample source 102 can include any corresponding gas sampling, handling, separation and/or treatment apparatus. Sample source 102 can include apparatus including but not limited to: ambient environment sampling apparatus, apparatus for generating a gas sample by treating solid and/or liquid samples, sample preparation apparatus, and gas separation apparatus (e.g., a gas chromatography apparatus).

The apparatus is configured to remove organic compounds that may be present in the initial gas sample such that the treated gas sample is substantially free of the organic compounds by maintaining the heating element at an elevated temperature (typically>400 C). The organic compounds are removed from the initial gas sample by oxidation and/or thermal decomposition due to the elevated temperature of the heating element. Importantly, the sample preparation chamber does not include any materials that are significantly altered in operation to remove the organic compounds. Preferably, analysis instrument 104 is an optical spectroscopic analysis instrument, although the present approach is applicable to sample preparation for any kind of gas analysis instrument. Suitable optical analysis instruments include, but are not limited to: cavity ringdown spectroscopy (CRDS) instruments and cavity enhanced absorption spectroscopy (CEAS) instruments. Suitable non-optical analysis instruments include, but are not limited to: mass spectrometry instruments.

The effectiveness of this approach has been confirmed in practice. For example, a concentration of organic compounds in the initial sample gas of 1 ppm/cm or less can be reduced to 0.01 ppm/cm or less in the treated sample gas (i.e., a 100× reduction of an already low initial contamination). Here ppm/cm is a unit of optical absorbance that is used as a convenient proxy for concentration. Propagation through 1 cm of a material having 1 ppm/cm absorbance gives an optical loss of 1 ppm.

In a preferred embodiment, the heating element is a Ni—Cr wire, which can readily be heated to high temperatures by resistive heating. Nickel-chromium wire is widely used as a heating element for devices such as space heaters and toasters. Targeted reproducible temperatures can be achieved by using the appropriate gauge, length, and geometry of the wire when a suitable direct current (or AC) voltage is applied. For example, 30-44 gauge Ni—Cr wire can reach suitable temperatures (>1000 C) when driven with 24 VDC. The wire can be heated up to the melting point of 1400 C, which is well above the temperatures required to chemically remove the interfering organic molecules by pyrolysis, oxidation and/or decomposition. An analyte such as water is stable up to 2000 C, so this temperature range will typically not affect analytes of interest.

Alternatively, inductive heating can be employed to heat the heating element by providing an external magnetic field. Inductive heating has the advantage that no electrical contacts to the heating element are required.

The sample preparation chamber is preferably configured such that gas molecules in the input sample gas are likely to collide at least once with the heating element during passage through the sample chamber. This concept can be made more precise by defining a residence time Tᵣₑₛ as the chamber volume divided by the gas flow rate. It is also helpful to define a diffusion time from a point X in the sample preparation chamber to the heating element as being the diffusion time from X to the point on the heating element that is closest to X. The largest of these diffusion times (as X varies over all points in the sample preparation chamber) is referred to as the maximum diffusion time Tₘₐₓ from the heating element to any point in the sample preparation chamber. Preferably, Tᵣₑₛ>Tₘₐₓ.

These considerations are helpful for determining preferred configurations for the sample preparation chamber. For example, it is apparent that all points in the sample preparation chamber are preferably relatively close to the heating element. One way to arrange this is to configure the sample preparation chamber as a shell surrounding a solid support, where the heating element is configured as a wire wrapped around the support. In this configuration, the gas flow region of the sample preparation chamber is an annular region between the support and the enclosing shell. Often, the direction of gas flow through such an arrangement is perpendicular to the coil loops (e.g., as shown on FIG. 1).

It is often preferred for the heating element to occupy a substantial fraction of the available volume in the sample chamber. For a shell that encloses a volume Ve and for a support that has volume Vs, the available volume between the shell and the support is Ve−Vs. In one example, the preferred design range for the heating element volume Vh is 0.39(Ve−Vs)<Vh<Ve−Vs.

Practice of the invention does not depend critically on whether or not the heating element acts as a catalyst at its surface. In some cases, it is expected that the heating element surface (e.g., NiO and/or CrO for a Ni—Cr element) can act as a catalyst. In other cases, it is expected that the heating element does not act as a catalyst. In either case, there is typically no other source of catalytic activity in the apparatus. In the first case, the sample preparation chamber does not include any catalytically active surface other than a surface of the heating element. In the second case, the sample preparation chamber does not include any catalytically active surface.

Optionally, a carrier gas source 106 can be connected to the input line to provide inert and/or oxidative carrier gas to the sample input chamber. In such cases, it is helpful to refer to the original sample gas (i.e., the gas emitted from source 102) as the “sample gas”, while the “input sample gas” is the gas provided to sample preparation chamber 108 (i.e., if a carrier gas is used, the “input sample gas” includes the carrier gas). An inert carrier gas can be helpful for establishing a controlled gas flow rate through the system. An oxidative carrier gas (e.g., oxygen) can be helpful to expedite oxidization of organic contaminants.

FIGS. 2a-b show two views of an first sample preparation chamber configuration. In this example, the heating element is configured as a wire 204 wrapped around a rectangular support 202. Support 202 is enclosed by a rectangular shell 206 having gas lines 210 and 212 for gas input and output, and having electrical connections 208 for resistive heating. The gas flow region of this example includes the annular region between support 202 and shell 206. Support 202 should be an electrical insulator capable of withstanding high temperatures (e.g., alumina).

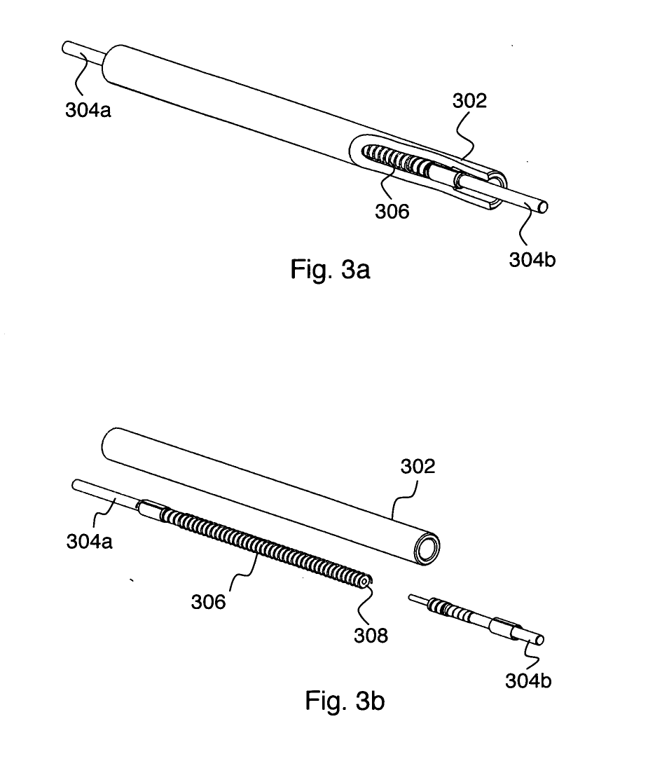

FIGS. 3a-b show two views of an second sample preparation chamber configuration. Here, FIG. 3a shows an assembled configuration and FIG. 3b shows an exploded view. In this example, the heating element is configured as a wire 306 wrapped around a cylindrical support 308. Support 308 is enclosed by a cylindrical shell 302. The gas flow region of this example includes the annular region between support 308 and shell 302. Electrical contacts 304a and 304b provide for resistive heating. Preferably, at least one of the electrical contacts is a spring loaded electrical contact, which improves manufacturability. In this example, contact 304b is shown as a spring-loaded contact. Support 308 should be an electrical insulator capable of withstanding high temperatures (e.g., alumina).

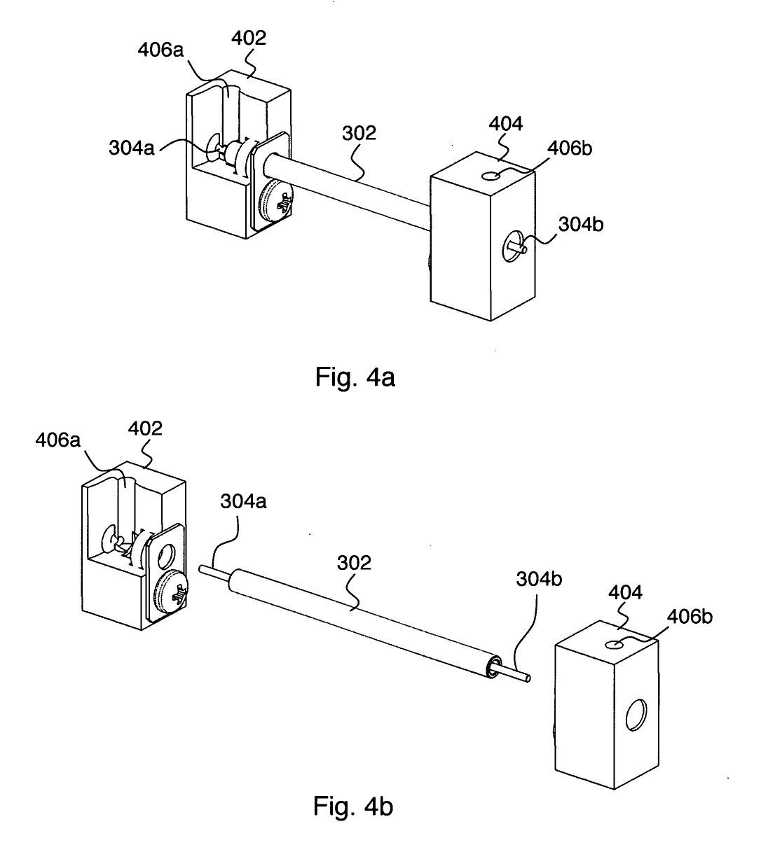

FIGS. 4a-b show two views of an assembly including the second sample preparation chamber configuration. Here, FIG. 4a shows an assembled configuration and FIG. 4b shows an exploded view. In this example, blocks 402 and 404 provide gas lines 406a and 406b respectively to the sample preparation chamber of FIGS. 3a-b.

1. Apparatus for gas sample analysis, the apparatus comprising:
a sample preparation chamber capable of receiving an input gas sample, wherein a heating element is disposed in the sample preparation chamber; and
an instrument for gas analysis configured to receive a treated gas sample from the sample preparation chamber;
wherein the apparatus is configured to remove organic compounds that may be present in the initial gas sample such that the treated gas sample is substantially free of the organic compounds by maintaining the heating element at an elevated temperature;
wherein the organic compounds are removed from the initial gas sample by oxidation and/or thermal decomposition due to an elevated temperature of the heating element; and
wherein the sample preparation chamber does not include any materials that are significantly altered in operation to remove the organic compounds.

2. The apparatus of claim 1, wherein the heating element comprises a Ni—Cr alloy.

3. The apparatus of claim 1, wherein the sample preparation chamber comprises an enclosing rectangular shell surrounding a rectangular support, wherein the heating element is wrapped around the rectangular support in a coil, and wherein a gas flow region of the sample preparation chamber includes an annular region between the rectangular support and the enclosing rectangular shell.

4. The apparatus of claim 1, wherein the sample preparation chamber comprises an enclosing tube surrounding a cylindrical support, wherein the heating element is wrapped around the cylindrical support in a coil, and wherein a gas flow region of the sample preparation chamber includes an annular region between the cylindrical support and the enclosing tube.

5. The apparatus of claim 1, wherein the heating element is wrapped around a support in a coil, and wherein a direction of carrier gas flow is substantially perpendicular to loops of the heating element coil.

6. The apparatus of claim 1, wherein the sample preparation chamber comprises an enclosing shell having enclosed volume Ve surrounding a support having volume Vs, wherein the heating element is wrapped around the support in a coil and has a volume of Vh, and wherein 0.39(Ve−Vs)<Vh<Ve−Vs.

7. The apparatus of claim 1, wherein gas flow through the sample preparation chamber defines a residence time for gas in the sample preparation chamber, and wherein the residence time is greater than a maximum diffusion time from the heating element to any point in the sample preparation chamber.

8. The apparatus of claim 1, wherein the heating element is resistively heated and wherein electrical contacts to the heating element are spring-loaded.

9. The apparatus of claim 1, wherein the heating element is configured to be inductively heated.

10. The apparatus of claim 1, wherein the sample preparation chamber does not include any catalytically active surface.

11. The apparatus of claim 1, wherein the sample preparation chamber does not include any catalytically active surface other than a surface of the heating element.

12. The apparatus of claim 1, wherein the gas analysis instrument comprises an optical spectroscopic gas analysis instrument.

13. The apparatus of claim 1, wherein the input gas sample is provided as a separated output from a gas chromatography apparatus.

14. A method of gas sample analysis, the method comprising:
flowing an input sample gas past a heating element disposed in a sample preparation chamber to provide a treated gas sample;
performing gas analysis of the treated gas sample with a gas analysis instrument; and
removing organic compounds that may be present in the initial gas sample such that the treated gas sample is substantially free of the organic compounds by maintaining the heating element at an elevated temperature;
wherein the organic compounds are removed from the initial gas sample by oxidation and/or thermal decomposition due to the elevated temperature of the heating element; and
wherein the sample preparation chamber does not include any materials that are significantly altered in operation to remove the organic compounds.

15. The method of claim 14, further comprising providing electrical current to the heating element such that the elevated temperature is provided by resistive heating.

16. The method of claim 14, further comprising providing a magnetic field to the heating element such that the elevated temperature is provided by inductive heating.

17. The method of claim 14, further comprising mixing an inert carrier gas to a sample gas to provide the initial sample gas.

18. The method of claim 14, further comprising mixing an oxidative carrier gas to a sample gas to provide the initial sample gas.

19. The method of claim 14, wherein the elevated temperature is greater than 400 C.

20. The method of claim 14, wherein a concentration of the organic compounds in the initial sample gas is 1 ppm/cm or less, and wherein a concentration of the organic compounds in the treated sample gas is 0.01 ppm/cm or less.

21. The method of claim 14, wherein gas flow through the sample preparation chamber defines a residence time for gas in the sample preparation chamber, and further comprising setting the flow conditions in the sample preparation chamber such that the residence time is greater than a maximum diffusion time from the heating element to any point in the sample preparation chamber.

22. The method of claim 14, further comprising separating an initial gas sample by gas chromatography to provide the input sample gas.
